(19) United States
(12) Patent Application Publication
(10) Pub. No.: US 2013/0194548 A1
(43) Pub. Date: Aug. 1, 2013

(54) PORTABLE RETINAL IMAGING DEVICE

(71) Applicant: RAYTHEON COMPANY, Waltham, MA (US)

(72) Inventors: Robert Paul Francis, Lewisville, TX (US); Lan Sun, Allen, TX (US)

(73) Assignee: RAYTHEON COMPANY, Waltham, MA (US)

(21) Appl. No.: 13/804,083

(22) Filed: Mar. 14, 2013

Related U.S. Application Data
(63) Continuation-in-part of application No. 13/440,464, filed on Apr. 5, 2012.
(60) Provisional application No. 61/472,986, filed on Apr. 7, 2011.
(60) Provisional application No. 61/491,502, filed on May 31, 2011.

(51) Int. Cl. A61B 3/15; A61B 3/12; A61B 3/14

(52) U.S. Cl. CPC A61B 3/15; A61B 3/14; A61B 3/12
USPC 351/208; 351/206; 351/246

(57) ABSTRACT

A portable MEMS-based scanning laser ophthalmoscope (MSLO). In one example the MSLO includes a laser illumination sub-assembly that generates a laser illumination beam, a two-dimensional MEMS scan mirror configured to receive and scan the laser illumination beam over at least a portion of the retina of an eye to be imaged, an optical system configured to direct the laser illumination beam from the scan mirror into the eye to illuminate the retina, and a detector sub-assembly configured to intercept optical radiation reflected from the eye to generate an image of the retina. The optical system includes a polarized beamsplitter positioned between the scan minor and the eye and configured to direct the laser illumination beam to into the eye and to direct the optical radiation reflected from the eye to the detector sub-assembly.

### CROSS REFERENCE TO RELATED APPLICATIONS

This application claims priority under 35 U.S.C. §120 to co-pending, commonly-owned U.S. application Ser. No. 13/440,464 titled “PORTABLE SELF-RETINAL IMAGING DEVICE” filed on Apr. 5, 2012, which claims priority under 35 U.S.C. §119(e) to U.S. Provisional Patent Application No. 61/472,986 titled “PORTABLE SELF-RETINAL IMAGING DEVICE” filed on Apr. 7, 2011 and to U.S. Provisional Patent Application No. 61/491,502 titled “PORTABLE SELF-RETINAL IMAGING DEVICE” filed on May 31, 2011, all of which are incorporated herein by reference in their entireties.

### BACKGROUND

Ophthalmic fundus cameras have been used by ophthalmic specialists for many years to image the interior surface of the eye (the retina), including the fundus, optic disc, macula and fovea, and posterior pole. Generally, a fundus camera has approximately a 30 to 45 degree spherical field of view on the retina. These cameras operate on the principle of direct or indirect ophthalmoscopy, and flood the eye with light from a flash bulb and capture a two-dimensional image with imaging optics and a detector. The light from the flash bulb is focused via a series of lenses through a doughnut-shaped aperture, and then passes through a central aperture to form an annulus before passing through the camera objective lens and through the cornea onto the retina. The light reflected from the retina passes through the un-illuminated hole in the doughnut formed by the illumination system to a telescopic eyepiece. To obtain an image of the retina, a mirror interrupts the path of the illumination system to allow the light from the flash bulb to pass into the eye, and simultaneously, a minor falls in front of the observation telescope to redirect the light onto the detector. These instruments are complex in design and difficult to manufacture to clinical standards. In addition, fundus cameras are limited to a relatively small field of view and worse than diffraction-limited resolution on the retina due to aberrations introduced by the imaging optics and the front objective common to both illumination and imaging paths. Portable or handheld fundus cameras are commercially available, but are not widely used because they require a skilled photographer for operation and the images captured are poor relative to tabletop devices.

Another device used to obtain images of the retina is the scanning laser ophthalmoscope, which is generally able to image the retina with better spatial resolution than a fundus camera. The scanning laser ophthalmoscope uses a laser illuminator which is raster scanned over the retina and a detector configured to measure light reflected from the retina at each point in the scan. The scanning elements used to scan the illuminator include rotating polygons, scanning prisms and galvanometer-driven movable mirrors. These elements are difficult to align and sensitive to shock and vibration, making their use impractical in portable systems.

For wide field of view (FOV) imaging, existing systems use an elliptical mirror for virtual point scanning in the retina. The real scan point is placed on one focus of the ellipse and the other focus of the ellipse is located in the pupil of the human eye. If the scan is symmetric about the minor axis of the ellipse, then the virtual scan angle is equal to the real scan angle.

### SUMMARY OF INVENTION

Aspects and embodiments are directed to a portable apparatus for obtaining an image of the retina. In particular, aspects and embodiments are directed to a scanning laser ophthalmoscope that replaces conventional scanning elements with a two-dimensional MEMS (microelectromechanical systems) scan mirror, thereby enabling robust scanning in a portable device, as discussed further below. According to certain embodiments, the device includes multi-color light sources with a polarization control unit for providing an incident polarized illumination, a two-dimensional MEMS scan mirror, and an optical imaging system for directing the illumination across the retinal surface. As discussed in more detail below, the scan mirror directs the incident illumination received along the optical axis and transmitted through the imaging system towards the retina of the eye. A polarized beamsplitter is used to direct image-bearing light reflected from the retina onto a confocal collection optical system and a detector, thereby obtaining an image of the retina.

According to one embodiment, a MEMS-based scanning laser ophthalmoscope comprises a laser illumination sub-assembly configured to generate a laser illumination beam, a two-dimensional MEMS scan minor configured to receive and scan the laser illumination beam over at least a portion of a retina of an eye to be imaged, an optical system optically coupled to the MEMS scan mirror and configured to direct the laser illumination beam from the scan minor into the eye to illuminate the retina of the eye, and a detector sub-assembly optically coupled to the optical system and configured to intercept optical radiation reflected from the eye to generate an image of the retina, wherein the optical system includes a polarized beamsplitter positioned between the scan minor and the eye and configured to direct the laser illumination beam to into the eye and to direct the optical radiation reflected from the eye to the detector sub-assembly.

In one example the two-dimensional MEMS scan minor is configured to scan the laser illumination beam over the portion of the retina in a Lissajous pattern. In one example the polarized beamsplitter is configured to transmit the laser illumination beam into the eye and to reflect the optical radiation reflected from the eye to the detector sub-assembly. In another example the polarized beamsplitter is configured to reflect the laser illumination beam into the eye and to transmit the optical radiation reflected from the eye to the detector sub-assembly. The optical system may further include an on-axis objective lens positioned between the polarized beamsplitter and the eye. The detector sub-assembly may include a photodetector, such as an avalanche photodiode, a charge coupled device, or a photo-multiplier tube, for example. In one example the detector sub-assembly further includes a focusing optic configured to focus the optical radiation to the photodetector. The detector sub-assembly may further include a confocal aperture optically coupled between the focusing optic and the photodetector. In another example the laser illumination sub-assembly includes at least one of a near-infrared laser source, and a visible laser source.

The MEMS-based scanning laser ophthalmoscope may further comprise a display screen optically coupled to the optical system, a controller configured to control the laser illumination sub-assembly to display a fixation target on the display screen, and a dichroic beamsplitter configured to optically couple the display screen into an illumination path along which the laser illumination beam travels to the eye, the illumination path including the polarized beamsplitter, and the polarized beamsplitter configured to direct light intensity corresponding to the fixation target into the eye to allow the eye to view the fixation target. In one example the controller is further configured to adjust a display location of the fixation target on the display screen to guide an orientation of the eye so as to obtain an image of a selected region of the retina. The MEMS-based scanning laser ophthalmoscope may further comprise an alignment and focus sub-system including an illuminator configured to provide an alignment beam, a camera configured to detect the alignment beam reflected from the eye, and a beamsplitter configured to couple the alignment beam into the illumination path. In another example the MEMS-based scanning laser ophthalmoscope further comprises an electrically tunable lens positioned in the illumination path between the laser illumination sub-assembly and the scan minor, wherein the controller is coupled to the camera and to the electrically tunable lens and is further configured to adjust a focus of the electrically tunable lens based on information obtained from the alignment beam reflected from the eye and detected by the camera.

According to another embodiment, a method of imaging a retina of an eye with a scanning laser ophthalmoscope comprises generating a laser illumination beam, directing the laser illumination beam to the eye with a polarized beamsplitter, scanning the laser illumination beam about a scan point at the eye using a two-dimensional MEMS scan mirror to produce a two-dimensional area of illumination that illuminates the retina of the eye, directing, with the polarized beamsplitter, optical radiation reflected from the eye to a detector sub-assembly without descanning the optical radiation, and producing an image of retina from the optical radiation.

In one example generating the laser illumination beam includes generating at least one of a near infra-red illumination beam and a visible illumination beam. In another example scanning the laser illumination beam includes scanning the laser illumination beam in a Lissajous pattern. In one example the polarized beamsplitter is positioned between the scan minor and the eye, and directing the laser illumination beam to the eye includes transmitting the laser illumination beam through the polarized beamsplitter, and directing the optical radiation reflected from the eye to the detector sub-assembly includes reflecting the optical radiation with the polarized beamsplitter. In another example the polarized beamsplitter is positioned between the scan minor and the eye, and directing the laser illumination beam to the eye includes reflecting the laser illumination beam with the polarized beamsplitter, and directing the optical radiation reflected from the eye to the detector sub-assembly includes transmitting the optical radiation through the polarized beamsplitter. The method may further comprise illuminating the eye with an alignment beam, detecting the alignment beam, and adjusting a focus of an electrically tunable lens positioned between a laser illumination sub-assembly that generates the laser illumination beam and the scan mirror to focus the laser illumination beam onto the retina of the eye. In one example the method further comprises displaying a fixation target on a display screen, and adjusting a display location of the fixation target on the display screen to guide an orientation of the eye so as to obtain an image of a selected region of the retina.

Still other aspects, embodiments, and advantages of these exemplary aspects and embodiments, are discussed in detail below. Any embodiment disclosed herein may be combined with any other embodiment in any manner consistent with at least one of the principles disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

### BRIEF DESCRIPTION OF THE DRAWINGS

Various aspects of at least one embodiment are discussed below with reference to the accompanying figures, which are not intended to be drawn to scale. The figures are included to provide illustration and a further understanding of the various aspects and embodiments, and are incorporated in and constitute a part of this specification, but are not intended as a definition of the limits of the invention. In the figures, each identical or nearly identical component that is illustrated in various figures is represented by a like numeral. For purposes of clarity, not every component may be labeled in every figure. In the figures:

FIG. 1 is a functional block diagram of one example of a MEMS-based scanning laser ophthalmoscope according to aspects of the invention;

FIG. 2 is a block diagram of one example of a portable MEMS-based scanning laser ophthalmoscope in a housing, according to aspects of the invention;

FIG. 3 is a schematic diagram illustrating an example of a conventional confocal scanning laser ophthalmoscope;

FIG. 4A is a schematic diagram illustrating one example of a transmission architecture for a MEMS-based scanning laser ophthalmoscope according to aspects of the invention;

FIG. 4B is a schematic diagram illustrating one example of a reflection architecture for a MEMS-based scanning laser ophthalmoscope according to aspects of the invention;

FIG. 5 is a functional block diagram of one example of a laser illumination sub-assembly according to aspects of the invention;

FIG. 6A is a schematic diagram of one example configuration of a MEMS-based scanning laser ophthalmoscope with a transmission architecture according to aspects of the invention;

FIG. 6B is a schematic diagram of one example configuration of a MEMS-based scanning laser ophthalmoscope with a reflection architecture according to aspects of the invention;

FIG. 7 is a schematic diagram of another example configuration of a MEMS-based scanning laser ophthalmoscope according to aspects of the invention;

FIG. 8 is a schematic diagram of another example configuration of a MEMS-based scanning laser ophthalmoscope according to aspects of the invention;

FIG. 9 is a schematic diagram of another example configuration of a MEMS-based scanning laser ophthalmoscope according to aspects of the invention;

FIG. 10 is a schematic diagram of another example configuration of a MEMS-based scanning laser ophthalmoscope according to aspects of the invention;

FIGS. 11A-C are a flow diagram of one example of a retinal imaging process according to aspects of the invention;

FIG. 12 is an example of an image of a display screen presented to a patient using an embodiment of the MEMS-based scanning laser ophthalmoscope, according to aspects of the invention;

FIG. 13 is a schematic diagram of one example configuration of a MEMS-based scanning laser ophthalmoscope configured to provide binocular fixation according to aspects of the invention; and

FIG. 14 is a schematic diagram of another example configuration of a MEMS-based scanning laser ophthalmoscope configured to provide binocular fixation according to aspects of the invention.

### DETAILED DESCRIPTION

Aspects and embodiments are directed to a compact, wide-field scanning laser ophthalmoscope configured to enable handheld, portable retinal imaging, for example, in remote locations and primary-care-physician offices. Portable retinal imaging would be invaluable for screening remote populations for eye disease, and for screening warfighters for ocular injury in the battlefield, to monitor immediate ocular effects of battlefield trauma. Similarly, retinal imaging in a physician's office would greatly improve the efficiency of screening diabetics for retinopathy, for example. Conventional table-top retinal imaging devices are too large for such applications and/or require a trained expert to operate.

According to one embodiment, self-administered, wide-field imaging of the retina in a compact, portable hardware footprint is achieved with a MEMS-based scanning laser ophthalmoscope (MSLO). To enable robust scanning in a portable device, a two-dimensional (2D) MEMS scanning minor replaces conventional scanning elements, such as the rotating polygons, scanning prisms and galvanometer-driven movable minors discussed above. According to certain embodiments, for a low-cost design, the off-axis conic minor front objective previously used in scanning laser ophthalmoscopes is replaced with an on-axis refractive objective lens, thereby reducing the complexity of the collection path and aberrations that have to be corrected in the illumination path. As discussed in more detail below, a polarized beamsplitter may be used to reduce ghost reflections caused by the use of refractive elements in the common illumination and detection/collection path. This allows for provision of a confocal scanning laser ophthalmoscope in which the collection path is not de-scanned, as discussed further below. In addition, embodiments of the MSLO are configured to present fixation targets to human subjects with real-time feedback to enable fully automated, self-administered retinal imaging, as also discussed further below.

Embodiments of the MSLO may enable retinal imaging outside of the traditional ophthalmologist office, including applications such as, for example, diabetic retinopathy screening using a telemedicine network, military ocular injury imaging in the field, retinal imaging in under-served locations of the world, and home care providers using portable systems. Another advantage of the MSLO is that it can be operated with low-light, so no pupil dilation is required.

It is to be appreciated that embodiments of the methods and apparatuses discussed herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiment. Also, the phraseology and terminology used herein is for the purpose of description and should not be regarded as limiting. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

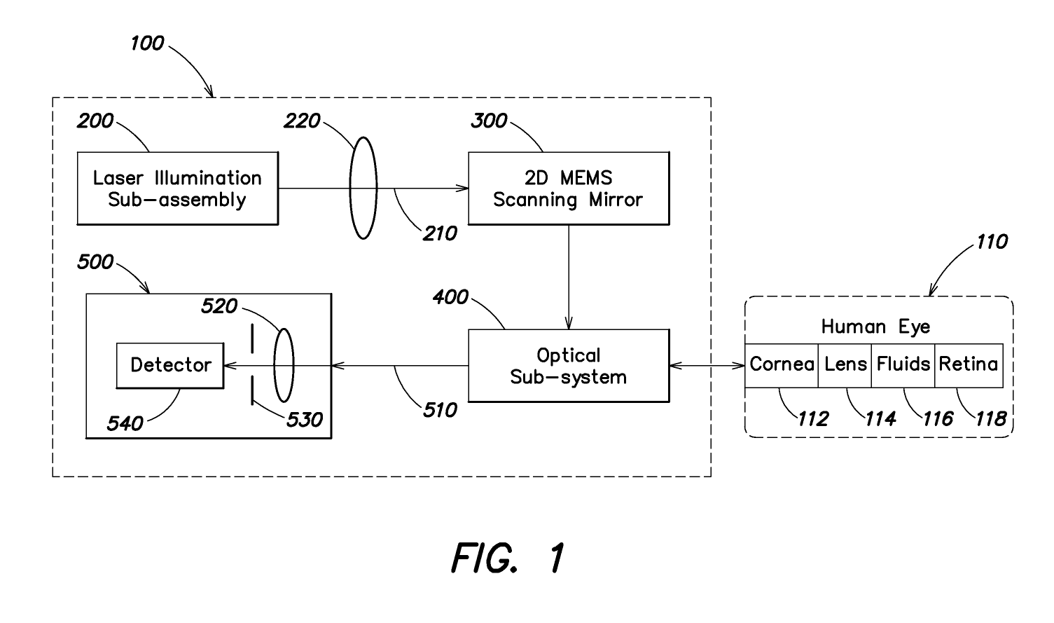

Referring to FIG. 1 there is illustrated a functional block diagram of one example of an MSLO according to one embodiment. As discussed in more detail below, the MSLO 100 may include one or more laser illumination sub-assemblies 200 that generate optical illumination beams 210 for scanning the human eye 110. In one example, the laser illumination sub-assembly 200 includes one or more lasers configured to generate the illumination beam(s) 210 at selected wavelengths, as discussed further below. The laser illumination sub-assembly 200 may include or may be coupled to focusing and/or collimating optics 220 to focus and/or collimate the illumination beam 210. The illumination beam 210 travels via an optical sub-system 400 through the cornea 112, the lens 114 and fluids 116 and is incident on the retina 118 of the eye 110. The illumination beam 210 is scanned across the retina 118 of the eye 110 using a MEMS scan minor 300. In one embodiment, the MEMS scan mirror 300 is a two-dimensional (2D) scan minor that scans the illumination beam(s) 210 in two dimensions over the retina 118, as discussed further below. Light scattered by the retina 118 travels back through the eye 110 and is directed by the optical sub-system 400 into a return beam 510 that is detected by an optical detector sub-assembly 500. In one example, the detector sub-assembly 500 includes focusing optics 520 and a confocal aperture 530 to direct and focus the return beam 510 onto a detector 540, as discussed further below. The detector 540 may be a photo-multiplier tube, avalanche photodiode, or charge-coupled device (CCD), for example.

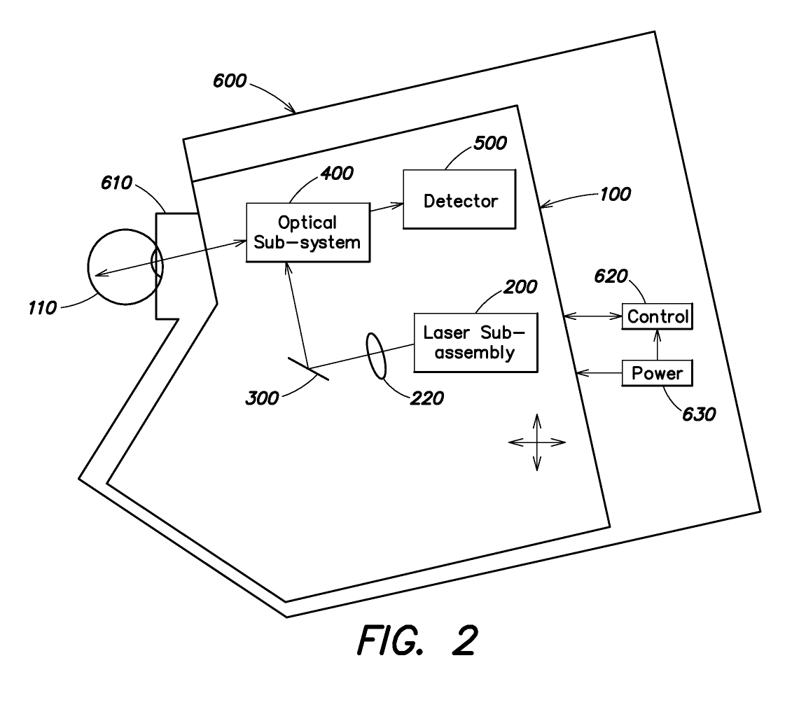

According to one embodiment, to enable automated, self-retinal imaging, the entire MSLO subsystem 100 is enclosed within a housing and electro-mechanically actuated to travel with six degrees of freedom to achieve best focus of the illumination beam 210 on the retina 118 of eye. FIG. 2 is a block diagram showing one example of the MSLO device with the optical subsystem arranged within a housing/enclosure 600. In one embodiment, the housing 600 includes an eyepiece portion 610 configured such that the patient can hold the device to their eye 110 to self-administer a retinal scan. The MSLO device may also include a controller 620 and a power supply 630 located within the housing 600. In one example the power supply 630 includes a battery. The power supply may provide power to any active components in the MSLO, including the laser illumination sub-assembly 200 for example, as well as to the controller 620.

The controller 620 may be configured to control various components and aspects of operation of the MSLO 100 to perform scanning of the patient's eye 110. For example, in embodiments in which one or more laser illumination sub-assemblies 200 include the ability to generate illumination beams 210 at different wavelengths, the controller 620 may control the wavelength(s) of light used for the illumination and/or the order in which beams of different wavelengths are scanned, as discussed further below. The controller 620 may further control any active components which may be included in the MSLO 100. The controller 620 may further control the processing, storage and/or transmission to a remote location of the output from the detector sub-assembly 500, as discussed further below. According to a variety of examples, the controller 620 includes a commercially available processor such as processors manufactured by Texas Instruments, Intel, AMD, Sun, IBM, Motorola, Freescale and ARM Holdings. However, the controller 620 may be any type of processor, field-programmable gate array, multiprocessor or controller, whether commercially available or specially manufactured.

The MSLO 100 may have any of numerous configurations (examples of which are discussed above) within the housing 600. In some embodiments, the physical structure and/or configuration of the housing 600, and/or the arrangement of the MSLO 100, controller 620 and power supply 630 within the housing, may affect the layout of the components of the MSLO, and optionally the optical configuration selected for the MSLO.

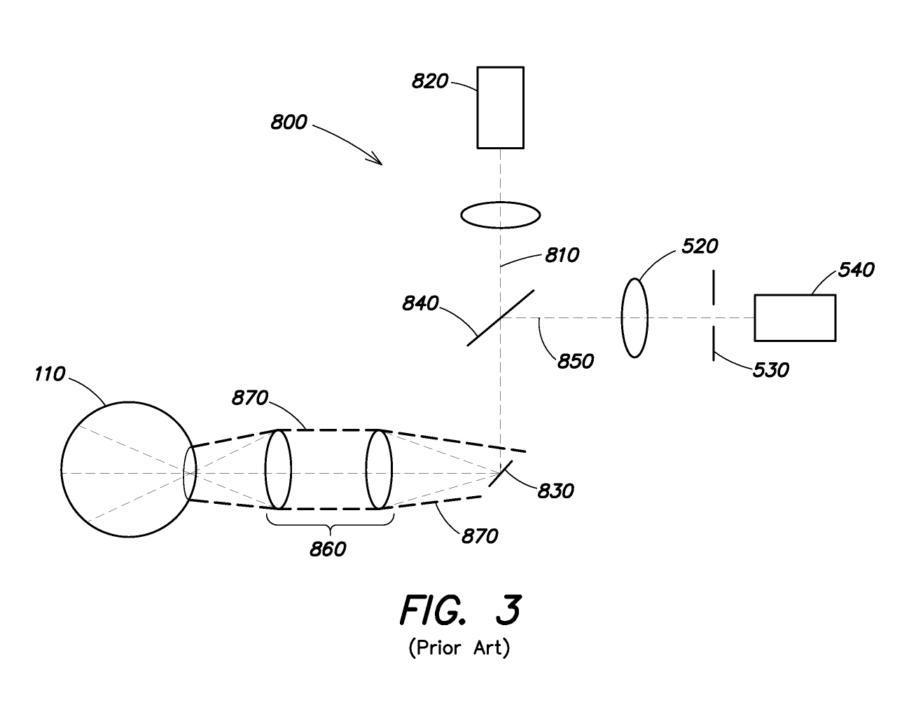

Referring to FIG. 3, in a conventional confocal scanning laser ophthalmoscope 800, the illumination beam 810 from an illuminator 820 is scanned by a scanning mirror 830, and the detection path is simultaneously de-scanned by the scanning mirror. A beam splitter 840 is used to separate the illumination path from the detection path, allowing the illumination beam 810 and the return beam 850 to share a portion of the same optical path, as shown in FIG. 3. A front objective 860 focuses the light to and from the eye 110 and scanning minor 830. Although the illumination beam 810 may have a relatively small beam diameter, light scattered from the retina back through a typical 3-5 mm diameter human pupil may increase significantly in diameter at the scanning minor due to beam magnification in the return path from the front objective 860. Dotted rays 870 represent the scattered rays from the retina of the eye 110. Thus, in this conventional configuration, since the scanning minor 830 is typically small, the free aperture of the scanning mirror may be significantly smaller than the beam diameter of the return beam. As a result, only a small portion of the light returning from the retina is reflected by the scanning minor 830 towards the detector sub-assembly 500.

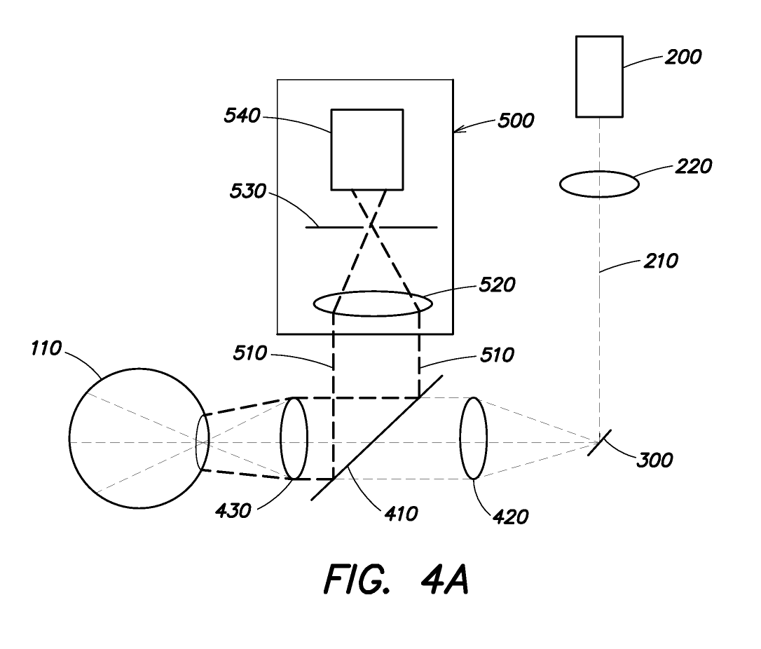

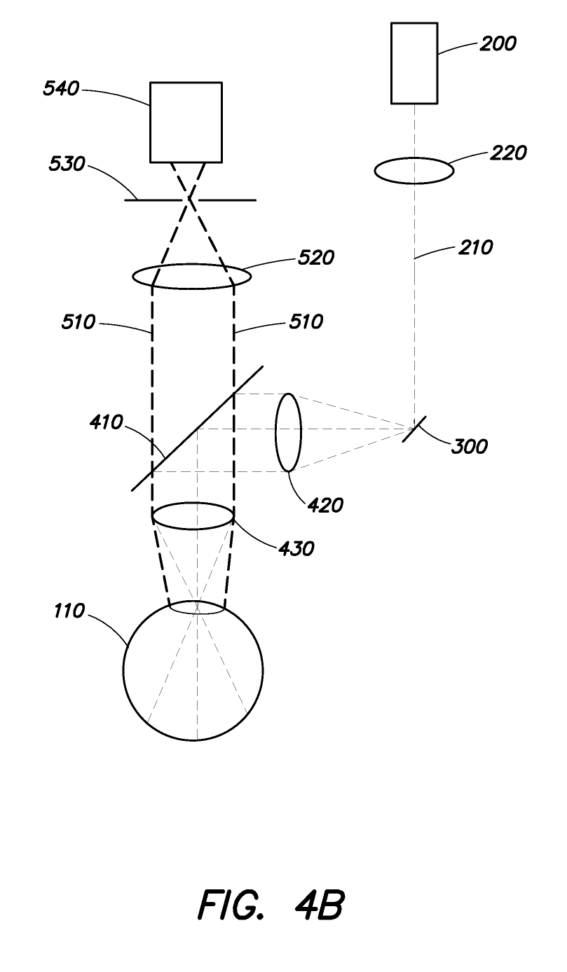

To address this issue and improve collection efficiency, aspects and embodiments provide a confocal MSLO in which the detection/collection path is not de-scanned. Rather, a beamsplitter is placed within the optical sub-system 400 to separate the illumination path and the collection path on the eye side of the scanning minor 300, as illustrated in FIGS. 4A and 4B. The beamsplitter 410 may be configured to either transmit the illumination beam 210 (and reflect the return beam 510), referred to herein as a transmission architecture (FIG. 4A), or reflect the illumination beam 210 (and transmit the return beam 510), referred to herein as a reflection architecture (FIG. 4B). According to certain embodiments, the optical sub-system 400 includes a scan lens 420 and a field lens 430. The beamsplitter 410 is positioned between the scan lens 420 and the field lens 430 to capture all of the returned light 510 from the retina and direct it to the detector sub-assembly 500. Thus, the beamsplitter 410 directs the return beam 510 to the detector sub-assembly 500 without de-scanning the return beam, and reduces the complexity of the collection path relative to some prior confocal scanning laser ophthalmoscope configurations.

As discussed above, in certain embodiments, the front objective for the MSLO (which is included in the optical sub-system 400) is implemented using one or more on-axis refractive elements, rather than an off-axis reflective conic objective. For example, in the embodiments shown in FIGS. 4A and 4B, the front objective includes the field lens 430. The use of an on-axis refractive front objective reduces the complexity of the collection path aberrations that have to be corrected in the illumination path, as well as reducing the size of the MSLO (by removing the need for large conic mirrors). However, the addition of a refractive element in the common illumination and collection path may increase ghost reflections which effectively create a bright spot in the center of detected images. In a conventional scanning laser ophthalmoscope arrangement, such as that illustrated in FIG. 3, for example, the detector 540 is placed at a fixed location behind the scanning minor 730 so that the light to be collected (from reflections from retina) are de-scanned back to the detector. In this configuration, ghost reflections may be blocked with an aperture stop at a single location. In contrast, as discussed above, embodiments of the MSLO discussed herein collect reflected light from the retina that is not de-scanned by placing the beamsplitter 410 near the eye 110. In other words, the light making up the return beam 510 is not reflected from the scan minor 300 after it has been reflected from the retina 118. As a result, ghost reflections as seen in the collection path do not appear at a single location.

According to one embodiment, the ghost reflections are greatly reduced by controlling the state of polarization in the illumination and collection paths such that the ghost reflections are polarized parallel to the illumination path and perpendicular to the collection path. In one embodiment, this polarization control is achieved by using a polarized beamsplitter 410. In one example, the polarized beamsplitter 410 is a cube beamsplitter configured to preferentially transmit P-polarized light and reflect S-polarized light. Thus, for example, referring to FIG. 4B, with the reflection architecture and assuming that the illumination beam is S-polarized, the polarized beamsplitter 410 reflects S-polarized light, and transmits orthogonal P-polarized light. The eye 110 is located in the reflection arm/path of the beamsplitter 410 such that the S-polarized light is incident on the refractive front objective (field lens 430) and the cornea of the eye. Ghost reflections maintain S-polarization, and are therefore not re-transmitted through the beamsplitter 410 to the detector sub-assembly 500. Reflections from the retina 118 are unpolarized since they are produced by multiple scatter events. Accordingly, some portion of the reflections from the retina are P-polarized and are re-transmitted through the beamsplitter 410 to form the return beam 510 at the detector sub-assembly 500. The reflection architecture may be preferred if the angle of the plane of incidence of light on the scan mirror 300 is parallel to the plane of incidence of the light on the polarized beamsplitter 410.

Similarly, referring again to FIG. 4A, with the transmission architecture since the ghost reflections are polarized parallel to the illumination path, they are largely transmitted through the beamsplitter 410 and not reflected to the detector sub-assembly 500. In some examples the transmission architecture may be preferred in configurations in which the angle of the plane of incidence of the light on the scan minor 300 is orthogonal to the plane of incidence of the light on the polarized beamsplitter 410 because the scan mirror, at 45 degrees, reflects S-polarized light more than P-polarized light. Thus, the polarized beamsplitter 410 forces the ghost reflections to a power level much lower than the power level of the reflections from the retina, effectively “blocking” or reducing the impact of the ghost reflections in the image.

As discussed above, in various embodiments of the MSLO 100 discussed herein, the scan minor 300 is a 2D MEMS micro-minor capable of high scanning speed and large angles of deflection. To illuminate a diffraction-limited spot on the retina, the beam entering the eye 110 must be approximately 1-2 millimeters (mm; 0.039-0.079 inches) in diameter, and nearly collimated. This beam diameter is determined by the optical properties of the human eye, with a 1 mm beam providing approximately the smallest/finest resolution in the eye. In one example, a 1 mm diameter beam at the cornea 112 enables a ten micron (0.0004 inches) spot size on the retina. In order to image a standard retinal field of view, for example, approximately 50 degrees, with minimal optical de-magnification of the illumination beam between the scan minor 300 and the cornea 112 of the eye, there must be minimal optical magnification of the scan angle between the scan minor and the cornea. This is because scan angle magnification from a curved front objective results in a corresponding beam diameter demagnification. Accordingly, it is desirable for the scan mirror 300 to have a large scan angle (range of angular motion) and high scan rate (speed) to allow scanning of the retinal field of view before eye movement distorts the image.

According to one embodiment, a two-dimensional scan of the retina 118 of a patient's eye 110 is performed by scanning the illumination beam 210 over the retina 118 in two dimensions. The 2D MEMS scan mirror 300 may be configured to implement a “raster” scan by “tilting” over its range of angular motion in both dimensions. In one example, the scan minor 300 has a fast dimension and a slow dimension, as is the case in conventional television raster scanning. However, the scan need not be rectangular; instead the scan mirror 300 may be configured to implement a spiral or vector raster scan.

In one example, the power supply 630 supplies a varying voltage to the 2D MEMS scanning minor 300 to activate the mirror to move over its range of angular motion (or selected portion thereof) to perform the scan. The scattered light from the retina 118 forms the return beams 510 which are collected by the detector sub-assembly 500. The detector 540 provides an output based on the detected return beams 510, and the output is processed to provide an image of retina. Image processing of the detector output may be performed, at least in part, by the controller 620. In one example, the controller 620 includes a storage device (not shown) for storing the detector output (raw or processed) to be provided to a remote user. For example, the controller may include a communications interface to transmit the detector output to a remote location for processing and/or analysis, or the storage may be removable from the housing 600 to allow the data stored thereon to be processed and/or analyzed on another machine. In one example, the storage includes non-transient computer-readable random access memory such as dynamic random access memory (DRAM), static memory (SRAM) or synchronous DRAM. However, the storage may include any device for storing data, such as non-volatile memory, with sufficient throughput and storage capacity to support the functions described herein.

In one embodiment, the scan minor 300 is implemented using state of the art advanced MEMS micro-mirror devices which are capable of large deflection angles to enable scanning over a field of view of approximately 35-50 degrees at a rate sufficiently fast to mitigate the effects of eye motion during the imaging scan. In one example, the 2D MEMS scan mirror 300 has a resonant frequency of greater than 23 kilohertz (kHz), is approximately 1.2 mm in diameter and has a mechanical deflection angle maximum (range of angular motion) of approximately 20 degrees peak-to-peak in each axis.

According to certain embodiments, the 2D MEMS micro-mirror is operated with both axes resonantly driven. In some cases in which both axes of the scan minor are resonant, the slow axis may not be slow enough for a raster scan to cover all field points in the field of view. Accordingly, a non-repeating Lissajous scan is used instead. A traditional raster scan pattern is generated when the fast scan axis frequency is an even multiple of the slow scan axis frequency. For large fields of view, in order to scan lines across the entire field of view, the slow scan axis frequency must be significantly lower than the fast scan axis frequency. In contrast, a Lissajous pattern is generated when the ratio of scan frequencies for the fast scan axis compared to the slow scan axis is an irrational number. For this pattern, the slow scan axis frequency need not be much lower than the fast scan axis frequency.

As discussed above, according to certain embodiments the laser illumination sub-assembly 200 of MSLO may include multiple lasers configured to generate illumination beams 210 at various different wavelengths. For example, near-infrared may be used for imaging the retina and visible light may be used to present a fixation target to a human subject, as discussed further below. The laser illumination sub-assembly 200 may be implemented using any of a variety of different types of lasers. The multiple illumination beams may also be generated by multiple individual laser illumination assemblies 200, or using an array of lasers within one or more laser illumination sub-assemblies. It is to be appreciated that various laser illuminators may be used, and that in any of the examples discussed and/or illustrated herein, one type or configuration of laser illumination sub-assembly may be replaced with another.

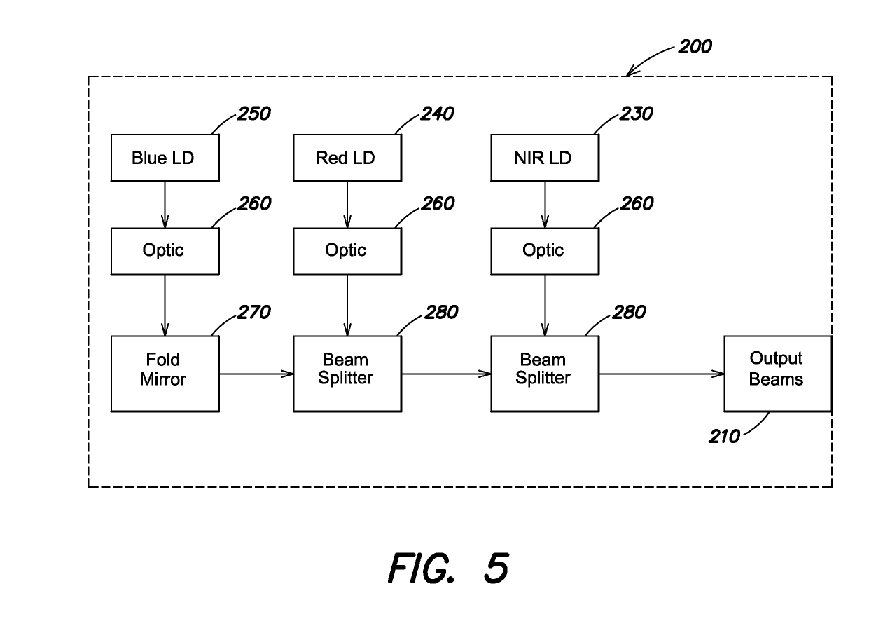

Referring to FIG. 5 there is illustrated a functional block diagram of one example of a laser illumination sub-assembly 200 according to one embodiment. The laser illumination sub-assembly 200 includes one or more lasers configured to generate the illumination beam(s) 210 at selected wavelengths. In one example, the MSLO may be configured for continuous near-infrared (NIR) retinal image acquisition and image processing, and accordingly in this example the laser illumination sub-assembly includes a near-infrared laser 230. Additional wavelengths may be useful for acquiring further information from the retinal scan and/or for implementing additional functionality in the MSLO. For example, visible illumination may be used for improved contrast of retinal vasculature and ischemia (e.g., green or orange-yellow laser illumination) and/or to provide a visible fixation image to the patient whose retina is being scanned, as discussed further below. Accordingly, the laser illumination sub-assembly may include one or more visible lasers, such as a red laser 240 and/or blue laser 250 as illustrated in FIG. 5. As used herein the term “visible laser” is intended to refer to a laser configured to emit a beam having a wavelength (or wavelength range) in the visible part of the electromagnetic spectrum. The lasers 230, 240 and 250 may be any type of suitable laser source, such as laser diodes or fiber lasers for example. Optics 260 may be used to collimate the output beams from the lasers 230, 240 and 250. In some embodiments, configuration of the laser packaging and/or arrangement of the laser illumination sub-assembly within the housing of the MSLO may result in one or more of the lasers not being directly in line with the desired pointing direction of the illumination beams 210. Accordingly, a fold minor 270 may be used to redirect the laser beams from one or more the lasers. Beam splitters 280 may be used to allow different lasers to share the same optical path. An additional beam splitter may be placed immediately in front of the scanning mirror so that the laser beam incident on the minor is normal to the mirrored surface when the mirror is at rest. This allows for a symmetric scan in four quadrants about the rest position, and reduces any geometric distortion caused by non-linear deflection angles in two-dimensions.

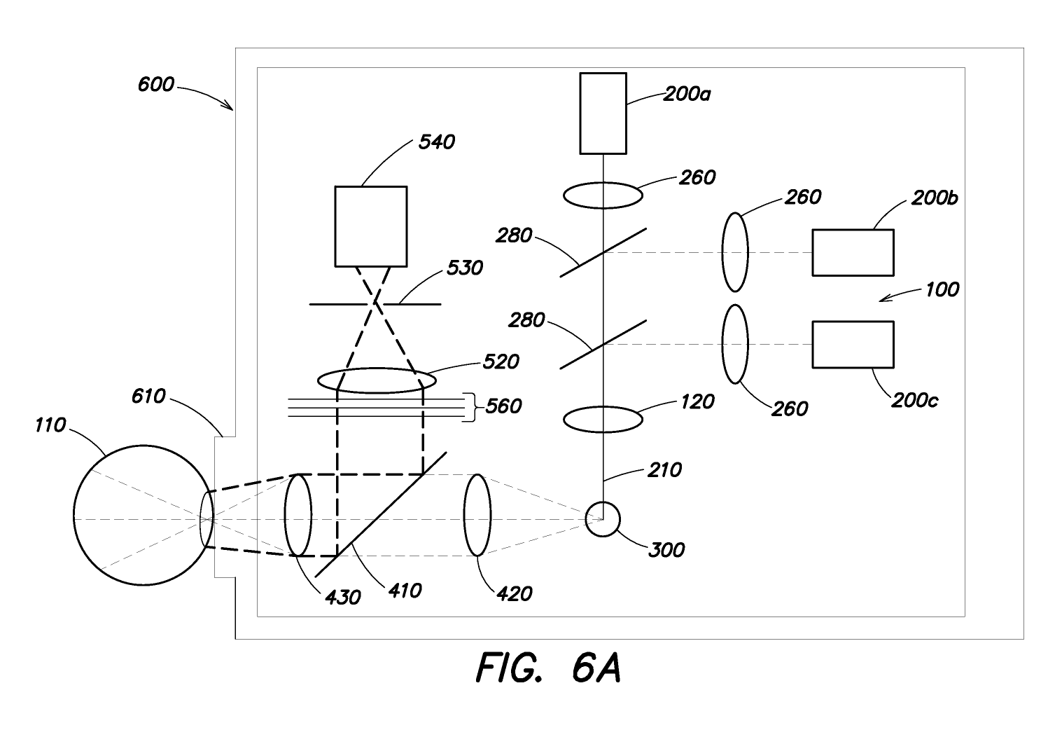

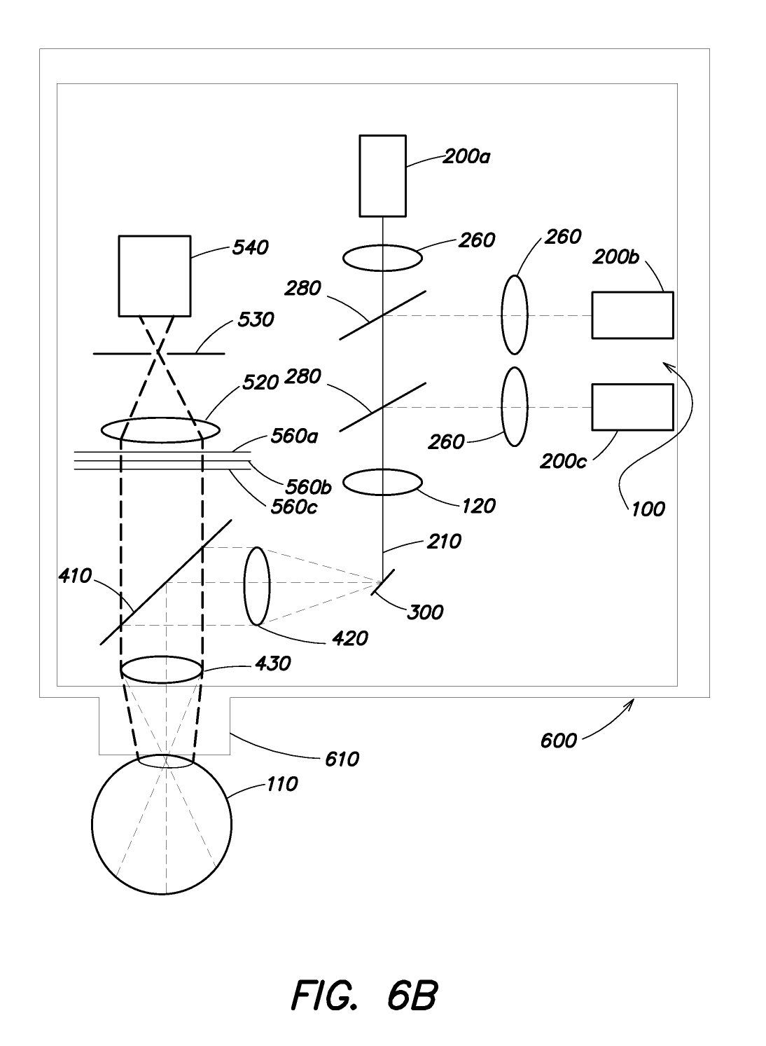

FIGS. 6A and 6B illustrate examples of an MSLO configured for multi-wavelength illumination with a transmission architecture and a reflection architecture, respectively. Each of the illuminators 200a, 200b, and 200c may include any one or more of the lasers 230, 240, and/or 250 discussed above and configured to laser at different wavelengths. Additionally, each of illuminators 200a, 200b, 200c may be individually and directly modulable. As discussed above, color-combining beamsplitters 280 may be used to combine the illumination beams of different wavelengths (colors) from each of the illuminators into the illumination beam 210. The detector sub-assembly may include, in the collection path as shown, one or more color filters 560 that selectively pass wavelengths of the return beam 510. In the illustrated example, the detector sub-assembly includes filters 560a-c, each of which may be matched to the wavelength (or wavelength range) of a corresponding illuminator 200a, 200b, and 200c.

The focusing optics 520 focus and direct the return beam 510 to the confocal aperture 530. The confocal aperture 530 may be used to filter light reflected from tissue layers outside of focal plane. As discussed above, the detector 540 may be any type of suitable photodetector including, for example, an avalanche photodiode, CCD or photo-multiplier tube. The output from the detector may be stored and/or provided to a processor, either integrated with the MSLO (e.g., controller 620) or remote, for analysis.

According to one embodiment, the MSLO includes an electrically tunable lens 120, which may be positioned in the illumination path between the laser illumination sub-assembly 200 and the scan mirror 300, as shown in FIGS. 6A and 6B. The electrically tunable lens 120 may be used for focus adjustment to better focus the illumination beam 210 on the retina 118 of the eye 110. In one example the electrically tunable lens 120 is an electro-tunable liquid lens, and may be adjusted or tuned under the control of the controller 620.

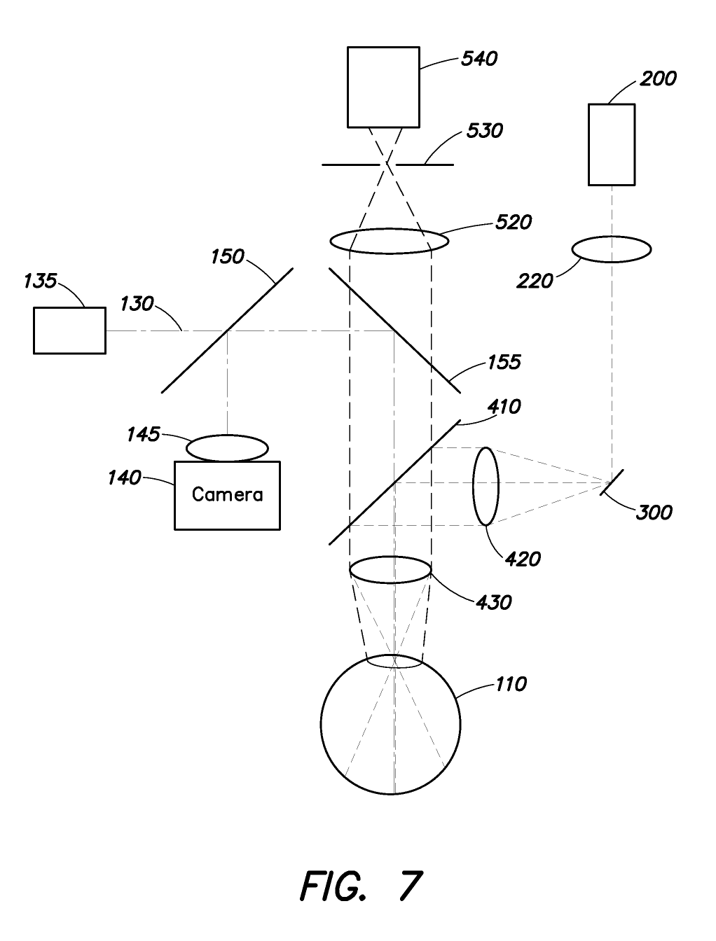

Referring to FIG. 7, in one embodiment the MSLO further includes a focus and alignment path, represented by dashed line 130. In this embodiment, the MSLO includes an illuminator 135, which may use an illumination source other than a laser, and which produces an alignment beam that is directed along the focus and alignment path 130 to and from the eye 110. An additional camera 140, optionally including associated focusing optics 145, may be included in the focus and alignment path 130. A beamsplitter 150 may be used to separate the forward and return paths, and to direct the returned alignment beam to the camera 140. In addition, a dichroic beamsplitter 155 may be used to separate the focus and alignment path 130 from the collection path. For example, where NIR radiation is used for retinal imaging, and the return beam 510 therefore includes NIR wavelengths, the alignment beam may be in the visible spectral band. Accordingly, the dichroic beamsplitter 155 may transmit the NIR radiation through to the detector sub-assembly 500 and reflect visible light to the beamsplitter 150. Alternatively, where visible light is used for the retinal imaging, the alignment beam may be an infrared beam, for example, an NIR beam, and the dichroic beamsplitter 155 may be selected to reflect NIR radiation and transmit visible light to the detector sub-assembly 500.

In one example, the camera 140 is coupled to the processor 620, which is coupled to the electrically tunable lens 120, and information from the focus and alignment path may be used to control the electrically tunable lens 120 to improve focus of the illumination beam 210 on the retina 118 of the eye 110. Information from the focus and alignment path 130 may also be used to adjust the positioning of optical elements of the MSLO to adjust the position of the scan point on the eye, and allow for different regions of interest on the retina to be imaged.

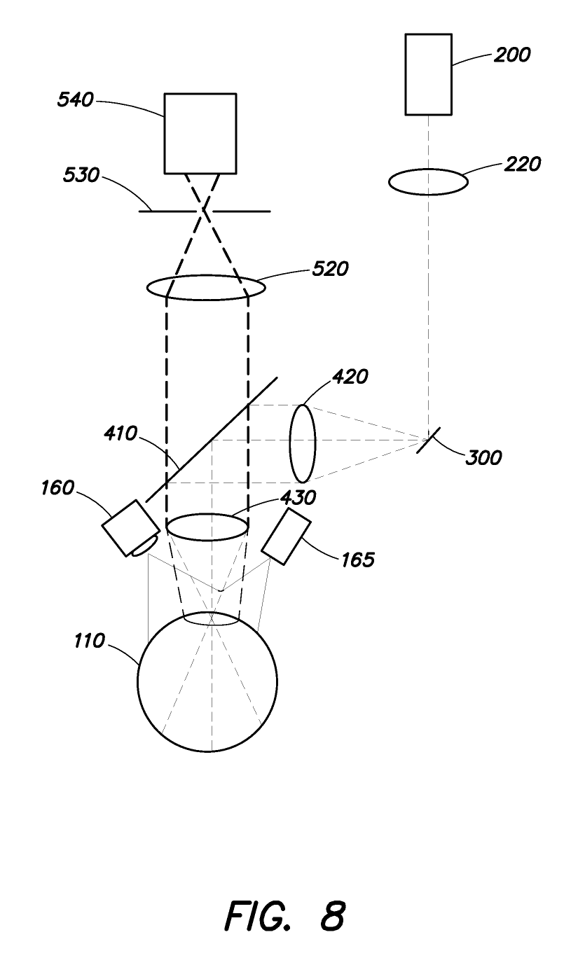

According to another embodiment, the MSLO 100 may include a camera 160 and illuminator 165 configured for continuous external eye imaging, as shown in FIG. 8. The camera 160 may be a miniature camera, so as not to add significant size or weight to the MSLO. The illuminator 165 may be an NIR illuminator, for example. The illuminator may be modulated or orthogonally polarized so as not to interfere with the MSLO imaging path.

During operation of the MSLO, a human subject peers through the eyepiece 610 and the MSLO continually scans illumination (for example, near-infrared, ˜780 nm) across the retina 118, and captures the response at the detector 540. As discussed above, an internal feedback loop, such as the focus and alignment path 130, may be used to adjust the position of the illumination beam on the retina 118. According to one embodiment, a fixation target is presented to the human subject to guide the subject's eye 110 to a desired location/angle to obtain images of certain areas of the retina 118. In one example, the fixation target is presented as an image formed by modulating a visible laser (e.g., ˜520 nm or ˜635 nm) at appropriate times in the scan. The image location may automatically adjust to guide the subject's eye to the best location for imaging, as discussed further below.

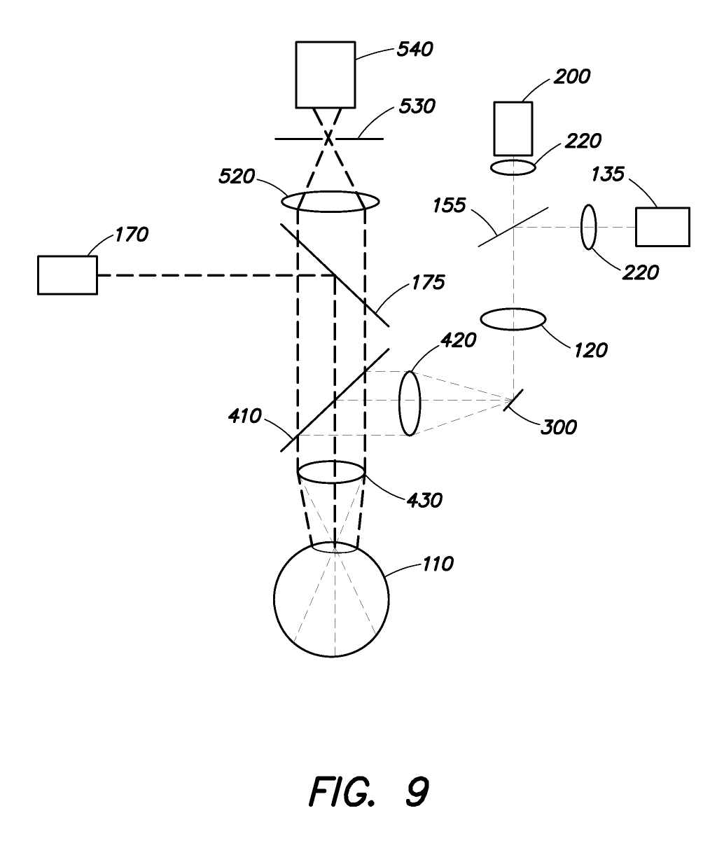

Referring to FIG. 9 there is illustrated one example of an MSLO configured to provide a fixation target to the human subject during the scan. The MSLO may include a display screen 170, for example, an LCD screen, which displays the fixation target. A dichroic beamsplitter 175 may be used to reflect the fixation target such that it is visible to the patient looking into the eyepiece 610 and can be used to guide the patient's viewing direction. A new retinal region may be imaged by adjusting the location of the fixation target on the display screen 170 and instructing the user to look at the new target location. Presentation of fixation targets with real-time feedback and optional audio instructions to the patient advantageously allows for fully automated, self-administered retinal imaging.

In the embodiment discussed above with reference to FIG. 7, the focus and alignment path is overlaid in the collection path. Alternatively, as illustrated in FIG. 9, the focus and alignment path may be coupled into the illumination path. In this case, the dichroic beamsplitter 155 is used to combine the illumination beam 210 and the alignment beam into a common optical path, as shown in FIG. 9. As discussed above, information from the focus and alignment path may be used to adjust an electrically tunable lens 120, and/or to adjust positioning of the fixation target to guide the patient's viewing direction.

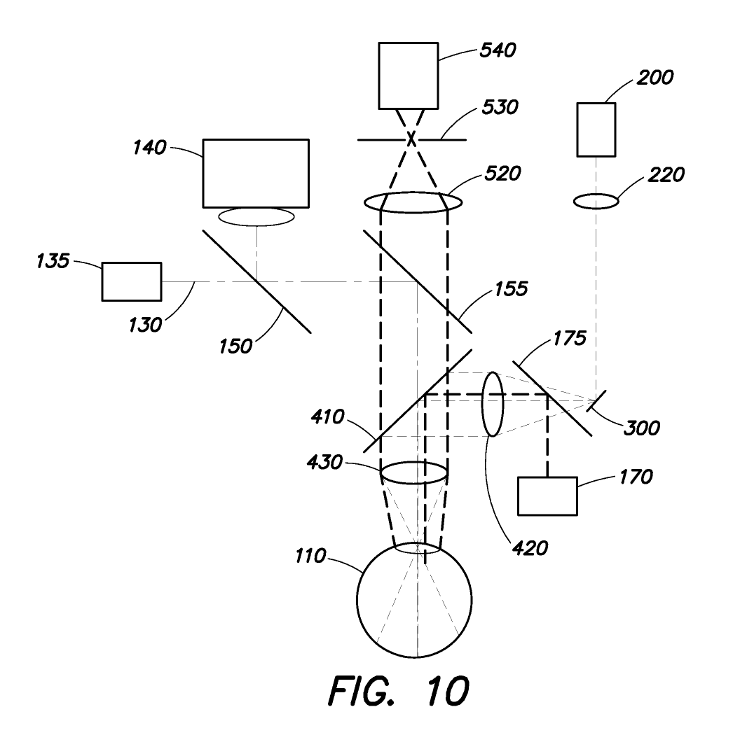

Another configuration of an MSLO including a fixation target displayed on display screen 170 and a focus and alignment path 130 is illustrated in FIG. 10. In this example, the fixation target is split into the illumination optical path using the beamsplitter 175. Alternatively, the beamsplitter 175 may be replaced with a Brewster window having a plane of incidence orthogonal to the plane of incidence of the illumination beam on the scan mirror 300.

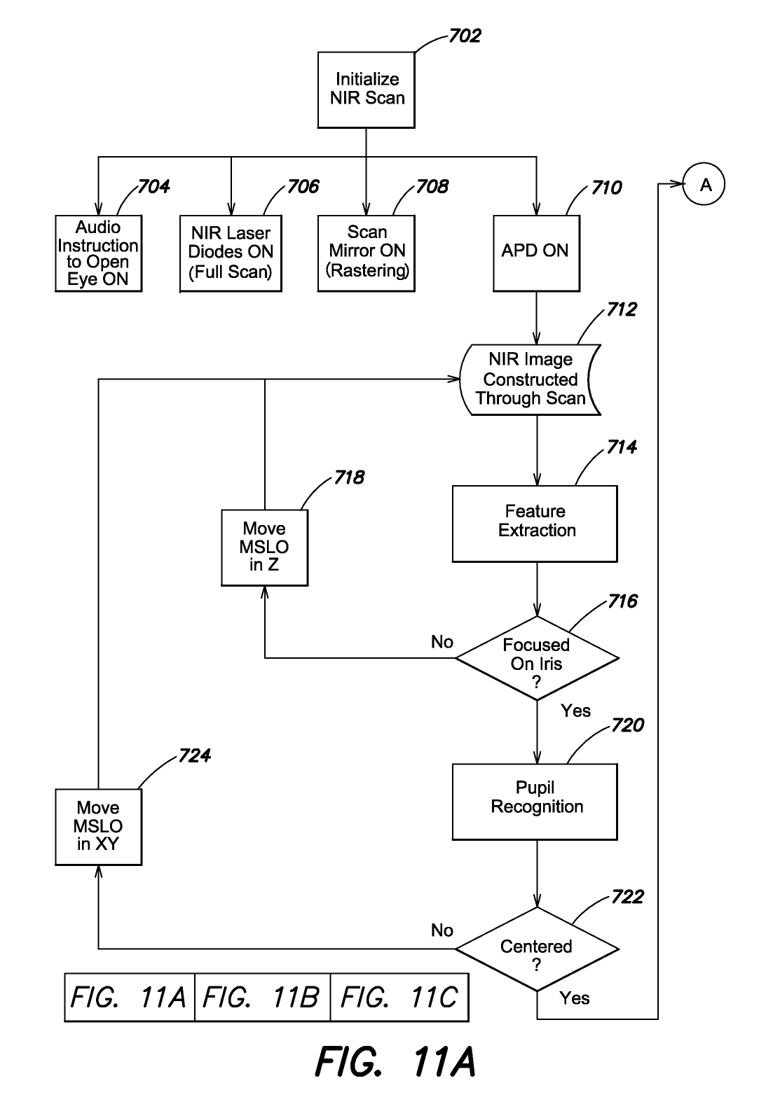

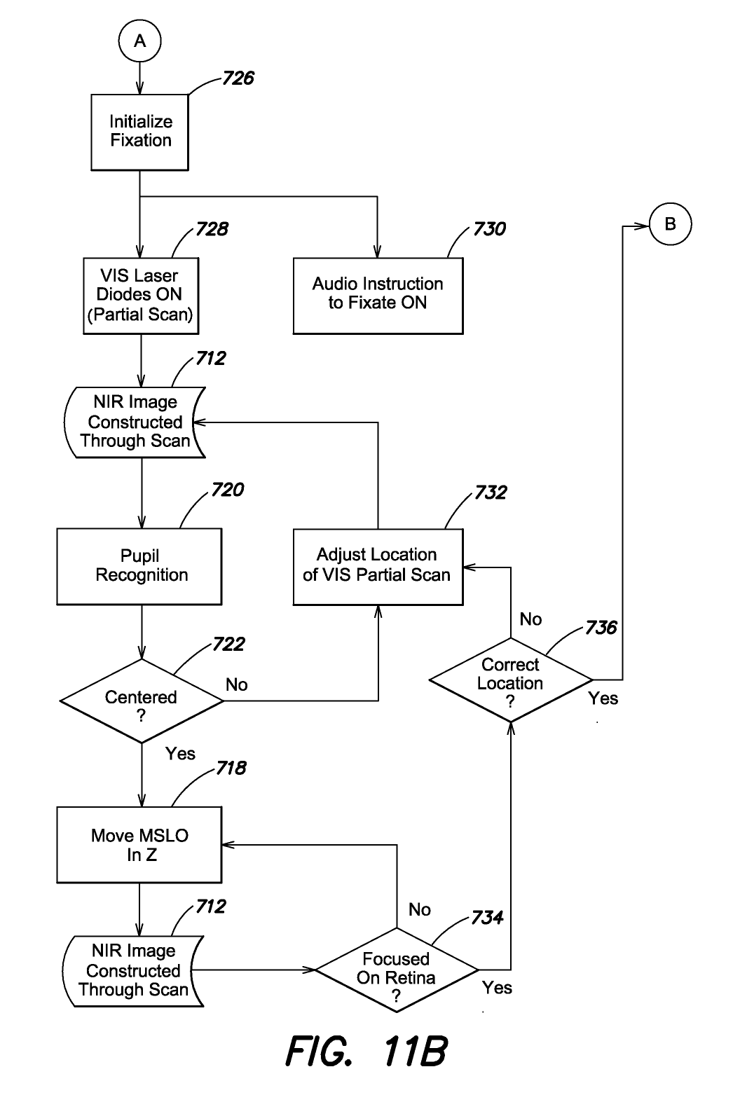

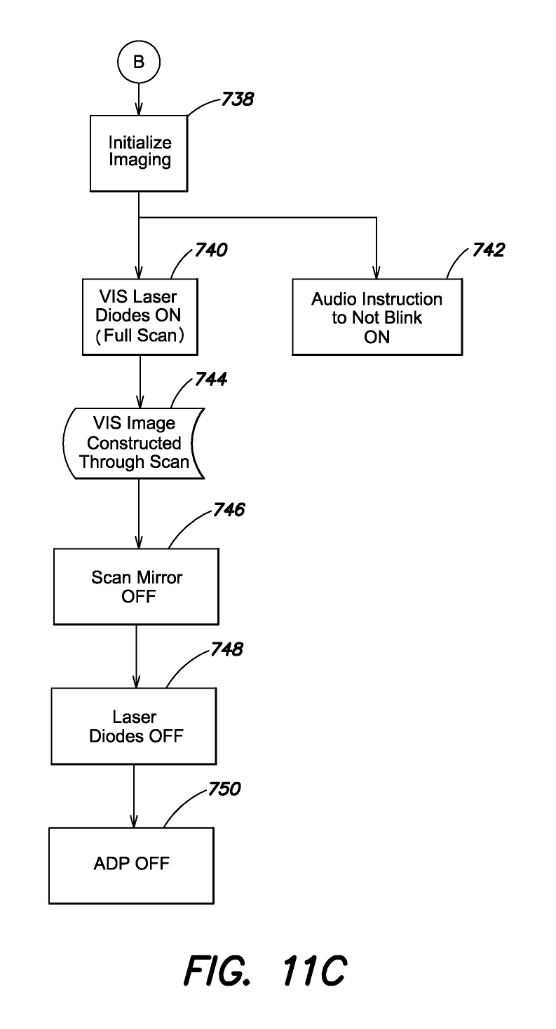

Referring to FIGS. 11A-C there is illustrated a flow diagram of one example of a retinal imaging process according to one embodiment. To begin a scan, a first step 702 may include initializing the scan at a desired wavelength. For example, an imaging scan of the retina 118 may be performed using a near-infrared laser as discussed above. Step 702 may begin when a user turns on the MSLO device, for example. Initializing the imaging scan may include instructing the patient to look into the eye piece and open their eye 110 (step 704). This instruction may be audible (for example, the MSLO device may include a speaker (not shown) and the controller 620 may direct the speaker to audibly project the instruction) and/or visual, as discussed further below. Initializing the imaging scan may also include turning on the laser illumination sub-assembly 200 and activating the laser at the desired wavelength (step 706), turning on the scan mirror 300 (step 708) and turning on the detector sub-assembly 500 (step 710). As discussed above, turning on the scan minor 300 (step 708) may include controlling the power supply 630 to provide a varying voltage to the 2D MEMS scanning minor to actuate the mirror to continuously move through its range of angular deflection in each dimension. As the 2D MEMS scan mirror moves, the illumination beam 210 is moved across the retina of the eye 110 to obtain an image of the retina (step 812).

According to one embodiment, the first scan after initialization (step 702) is used to determine whether the patient's eye 110 is oriented correctly for imaging a desired region of the retina and whether the eye is in focus. Accordingly, the controller 620 may process the image obtained in step 712, for example, by performing feature extraction processing (step 714) to locate specific points in the image, for example the iris, the pupil and/or portions of the retina. Following the feature extraction processing (step 714), the controller may determine whether or not the illumination beam is focused on the iris of the eye 110 (step 716). If the iris is not in focus, the controller may move the MSLO 100 (or certain optical components thereof) in the z-direction (step 718). In one embodiment, the MSLO 100 may be mounted on movable linear stages within the housing 600 to allow movement of the MSLO (or at least certain optical components thereof) in the x-, y- and z-axes (forward and back, left and right, up and down). After moving the MSLO in step 718, a new image may be obtained in step 712 and processed in step 714 to determine whether or not the iris is now in focus (step 716). This process may be repeated until the iris is correctly focused in the image.

The controller 620 may then process the image to locate the pupil of the eye 110 in the image (step 720) and determine whether or not the pupil is centered (step 722). If the pupil is not centered, the controller 620 may control the movable linear stages discussed above to move the MSLO 100 along the x- and/or y-axes (step 724) until the pupil is centered in the image.

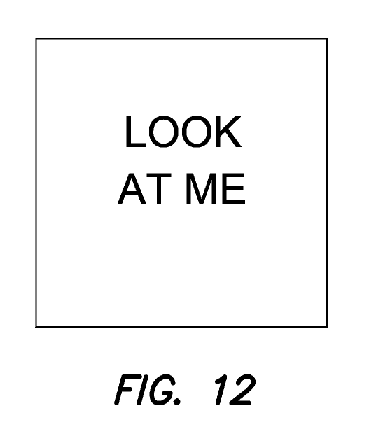

After initial set-up has been completed, the system may be configured to perform one or more scans of desired regions of the retina, using presentation of fixation targets with real-time feedback to enable fully automated, self-administered retinal imaging. As discussed above, in one example, the retinal image is obtained using infrared illumination. At the same time as the infrared scan is being performed, visible illumination is modulated appropriately to draw a fixation target at appropriate locations so that the human eye is oriented correctly to image the desired region of the retina. Thus, referring to FIGS. 11A-C, in one embodiment, step 726 includes initializing the fixation process, including activating one or more visible lasers to project the fixation target (step 728) and projecting an audible instruction to the patient to look at the fixation target (step 730). During the initial infrared set-up scanning discussed above, the fixation display 170 may be blank. When the fixation process is activated, the fixation target is displayed on the screen 170, for example, as illustrated in FIG. 12.

Initially when the patient looks into the eyepiece 610, the eye 110 may not be oriented correctly, and accordingly the image obtained of the eye may be off-center. In one embodiment, the system is configured to obtain an image of the eye 110, e.g., using infrared illumination as discussed above, (step 712), recognize the pupil in the image (step 720) and determine whether the pupil is centered (step 722). If the pupil is not centered, the controller 620 may control the system to adjust the location of the fixation target (step 732) until the eye is oriented such that the pupil is centered in the image. The controller 620 may then analyze the image to determine whether or not the retina is in focus (step 734) and move the MSLO or an internal focusing optic in the z-direction (step 718) until the retina is in focus. In some instances, the retina may be in focus, but the area of interest may not be in view. Accordingly, the controller 620 may determine whether or not the correct area of the retina is visible (step 736) and if not, move the fixation target to induce eye movement. The MSLO, or certain optical components thereof, may be moved laterally to compensate for the eye 110 tracking the fixation target.

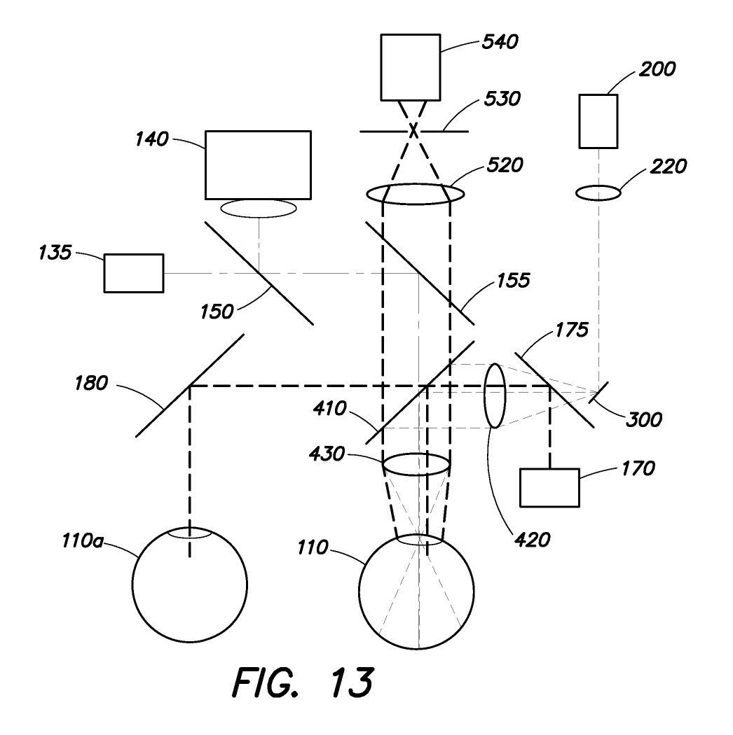

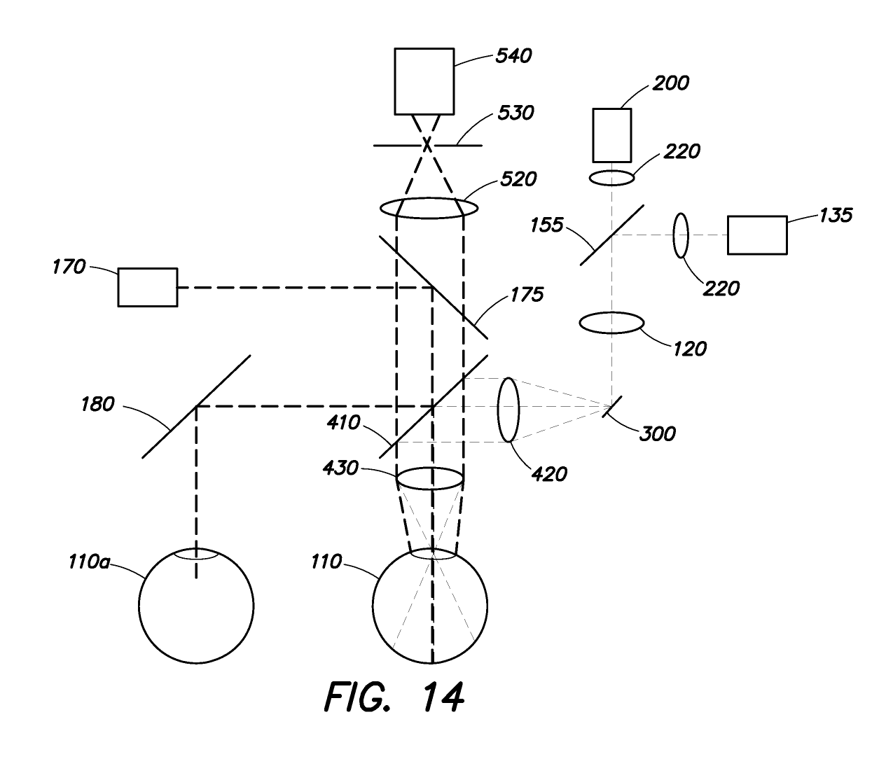

According to one embodiment, the MSLO 100 is configured to provide binocular fixation (i.e., the fixation target is presented to both eyes of the human patient). FIG. 13 illustrates one example of a configuration providing binocular fixation. A percentage of the light intensity of the fixation target displayed on the display screen 170 is transmitted through the polarized beamsplitter 410 and reflected from a fold minor 180 into the non-imaged eye 110a. The remainder of the light intensity of the fixation target is transmitted through the polarized beamsplitter 410 into the imaged eye 110, as discussed above. This forces both eyes to look in the same direction, thus reducing the likelihood of eye movement due to muscular confusion. FIG. 14 illustrates another MSLO configuration providing binocular fixation.

Referring again to FIGS. 11A-C, according to one embodiment, once the set-up has been completed and the correct area of the retina is in focus, the MSLO may be initialized (step 738) to perform one or more scans to obtain image(s) of the retina. These scans may use infrared and/or visual illumination and accordingly, the lasers to be used may be turned on (if not already on) and configured to perform a full scan (step 740). In one example, the system may project an audio instruction to the patient to not blink during the scan (step 742). The images are obtained by scanning the illumination beams across the retina using the 2D MEMS scan minor 300, as discussed above (step 744). The image(s) of interest may be stored for manual or automated analysis. Various steps of the process may be repeated to obtain images of different areas of the retina and/or at different wavelengths to provide different information about the patient's retina. When all scans are complete, the system may be shut down, including powering off the scan mirror 300 (step 746), the laser illumination sub-assembly 200 (step 748) and the detector sub-assembly (step 750).

Thus, aspects and embodiments provide a compact wide-field scanning laser ophthalmoscope using a 2D MEMS micro-mirror for fast scanning with few moving parts and robust portability in a light-weight package. Having all optics on-axis advantageously reduces aberrations. Although the inclusion of common path lenses may lower the signal-to-noise ratio in some circumstances, as discussed above, control of the illumination and detection polarization through the use of a polarized beamsplitter may negate the negative effect of common path lenses. Thus, wide field of view (e.g., approximately 50 degrees) and broadband color correction (for example, over a range of approximately 450 nm to 800 nm) are provided in a lightweight package. In addition, adjustable fixation targets may be presented to human subjects with real-time feedback to enable fully automated (including auto-alignment, auto-focus, and auto-capture/image acquisition), self-administered retinal imaging, as discussed above. Embodiments of the MSLO may make possible self-administered retinal imaging in any location, allowing for earlier diagnosis of eye disease, which will reduce blindness and improve worldwide health.

Having described above several aspects of at least one embodiment, it is to be appreciated various alterations, modifications, and improvements will readily occur to those skilled in the art given the benefit of this disclosure. Such alterations, modifications, and improvements are intended to be part of this disclosure and are intended to be within the scope of the invention. Accordingly, the foregoing description and drawings are by way of example only, and the scope of the invention should be determined from proper construction of the appended claims, and their equivalents.

What is claimed is:

1. A MEMS-based scanning laser ophthalmoscope comprising:
a laser illumination sub-assembly configured to generate a laser illumination beam;
a two-dimensional MEMS scan minor configured to receive and scan the laser illumination beam over at least a portion of a retina of an eye to be imaged;
an optical system optically coupled to the MEMS scan mirror and configured to direct the laser illumination beam from the scan minor into the eye to illuminate the retina of the eye; and
a detector sub-assembly optically coupled to the optical system and configured to intercept optical radiation reflected from the eye to generate an image of the retina;
wherein the optical system includes a polarized beamsplitter positioned between the scan mirror and the eye and configured to direct the laser illumination beam to into the eye and to direct the optical radiation reflected from the eye to the detector sub-assembly.

2. The MEMS-based scanning laser ophthalmoscope of claim 1, wherein the two-dimensional MEMS scan mirror is configured to scan the laser illumination beam over the portion of the retina in a Lissajous pattern.

3. The MEMS-based scanning laser ophthalmoscope of claim 1, wherein the polarized beamsplitter is configured to transmit the laser illumination beam into the eye and to reflect the optical radiation reflected from the eye to the detector sub-assembly.

4. The MEMS-based scanning laser ophthalmoscope of claim 1, wherein the polarized beamsplitter is configured to reflect the laser illumination beam into the eye and to transmit the optical radiation reflected from the eye to the detector sub-assembly.

5. The MEMS-based scanning laser ophthalmoscope of claim 1, wherein the optical system further includes an on-axis objective lens positioned between the polarized beamsplitter and the eye.

6. The MEMS-based scanning laser ophthalmoscope of claim 1, wherein the detector sub-assembly includes a photodetector, the photodetector comprising one of an avalanche photodiode, a charge coupled device, and a photo-multiplier tube.

7. The MEMS-based scanning laser ophthalmoscope of claim 6, wherein the detector sub-assembly further includes a focusing optic configured to focus the optical radiation to the photodetector.

8. The MEMS-based scanning laser ophthalmoscope of claim 7, wherein the detector sub-assembly further includes a confocal aperture optically coupled between the focusing optic and the photodetector.

9. The MEMS-based scanning laser ophthalmoscope of claim 1, wherein the laser illumination sub-assembly includes at least one of a near-infrared laser source, and a visible laser source.

10. The MEMS-based scanning laser ophthalmoscope of claim 1, further comprising:
a display screen optically coupled to the optical system;
a controller configured to control the laser illumination sub-assembly to display a fixation target on the display screen; and
a dichroic beamsplitter configured to optically couple the display screen into an illumination path along which the laser illumination beam travels to the eye, the illumination path including the polarized beamsplitter, and the polarized beamsplitter configured to direct light intensity corresponding to the fixation target into the eye to allow the eye to view the fixation target.

11. The MEMS-based scanning laser ophthalmoscope of claim 10, wherein the controller is further configured to adjust a display location of the fixation target on the display screen to guide an orientation of the eye so as to obtain an image of a selected region of the retina.

12. The MEMS-based scanning laser ophthalmoscope of claim 10, further comprising an alignment and focus sub-system including:
an illuminator configured to provide an alignment beam;
a camera configured to detect the alignment beam reflected from the eye; and
a beamsplitter configured to couple the alignment beam into the illumination path.

13. The MEMS-based scanning laser ophthalmoscope of claim 12, further comprising:
an electrically tunable lens positioned in the illumination path between the laser illumination sub-assembly and the scan mirror;
wherein the controller is coupled to the camera and to the electrically tunable lens and is further configured to adjust a focus of the electrically tunable lens based on information obtained from the alignment beam reflected from the eye and detected by the camera.

14. A method of imaging a retina of an eye with a scanning laser ophthalmoscope, the method comprising:
generating a laser illumination beam;
directing the laser illumination beam to the eye with a polarized beamsplitter;
scanning the laser illumination beam about a scan point at the eye using a two-dimensional MEMS scan minor to produce a two-dimensional area of illumination that illuminates the retina of the eye;
directing, with the polarized beamsplitter, optical radiation reflected from the eye to a detector sub-assembly without descanning the optical radiation; and
producing an image of retina from the optical radiation.

15. The method of claim 14, wherein generating the laser illumination beam includes generating at least one of a near infra-red illumination beam and a visible illumination beam.

16. The method of claim 14, wherein scanning the laser illumination beam includes scanning the laser illumination beam in a Lissajous pattern.

17. The method of claim 14, wherein the polarized beamsplitter is positioned between the scan minor and the eye, and wherein directing the laser illumination beam to the eye includes transmitting the laser illumination beam through the polarized beamsplitter, and directing the optical radiation reflected from the eye to the detector sub-assembly includes reflecting the optical radiation with the polarized beamsplitter.

18. The method of claim 14, wherein the polarized beamsplitter is positioned between the scan minor and the eye, and wherein directing the laser illumination beam to the eye includes reflecting the laser illumination beam with the polarized beamsplitter, and directing the optical radiation reflected from the eye to the detector sub-assembly includes transmitting the optical radiation through the polarized beamsplitter.

19. The method of claim 14, further comprising:
illuminating the eye with an alignment beam;
detecting the alignment beam; and
adjusting a focus of an electrically tunable lens positioned between a laser illumination sub-assembly that generates the laser illumination beam and the scan minor to focus the laser illumination beam onto the retina of the eye.

20. The method of claim 14, further comprising:
displaying a fixation target on a display screen; and
adjusting a display location of the fixation target on the display screen to guide an orientation of the eye so as to obtain an image of a selected region of the retina.
